(19) United States
(12) Patent Application Publication
(10) Pub. No.: US 2014/0150133 A1
(43) Pub. Date: May 29, 2014

(54) Use of Modified OsbZIP46 Gene in Controlling Plant Drought Resistance

(71) Applicants: Lizhong Xiong, Wuhan (CN); Ning Tang, Wuhan (CN)

(72) Inventors: Lizhong Xiong, Wuhan (CN); Ning Tang, Wuhan (CN)

(73) Assignee: HUAZHONG AGRICULTURAL UNIVERSITY, Wuhan (CN)

(21) Appl. No.: 14/112,073

(22) PCT Filed: Apr. 20, 2012
(86) PCT No.: PCT/CN2012/074476

§ 371 Date: Jan. 29, 2014

(30) Foreign Application Priority Data
Apr. 21, 2011 (CN) 201110099683.7

(51) Int. Cl. C12N 15/82

(52) U.S. Cl. CPC C12N 15/8273
USPC 800/278; 536/23.6; 435/320.1; 800/298; 800/320.1; 800/314; 800/312; 800/320.2; 800/320.3

(57) ABSTRACT

A genetically modified OsbZIP46CA1 gene is provided. Use of the modified gene in controlling drought resistance of a plant such as rice, and plants and cells comprising the modified gene are also provided.

### TECHNICAL FIELD

The present invention relates to the field of genetic engineering of plants, in particular to modified OsbZIP46 gene, a member of rice bZIP family and use of the modified gene in genetic improvement for rice drought resistance.

### BACKGROUND ART

Rice is one of the most important food crops in the world and with the worsening environmental problems, rice production is now facing a plurality of challenges, one of which is the effects of various abiotic stresses on rice growth and development. Nowadays, shortage of water resource and soil salinization are global problems limiting agricultural production since a lot of fields are dry or semiarid regions and many fields are threatened by salinity. Under natural conditions, environmental stress severely affects growth and development of crops such as rice, inhibits expression of their genetic potential and reduces crop yield, worsening ecological environments. Thus, improving rice resistance to drought and salinity has been one of urgent key problems to be solved in modern plant research. It seems more important to improve rice variety in term of stress resistance using transgenic technology and the key issue thereof is to screen genes useful in genetically improving stress resistance in rice. In plants, there are complicated stress response systems, in which a lot of genes are involved, including regulatory genes and some downstream functional genes, etc. Transcriptional factors are of great importance in regulation of stress response of plants, wherein bZIP family is one critical category (Jakoby et al, bZIP transcription factors in Arabidopsis. Trends Plant Sci. 2002 March; 7(3):106-11). In plants, bZIP transcriptional factors contain basic leucine zipper domain and are widely involved in response to abiotic stress through ABA dependent pathways. In plants, ABA synthesis can be induced by abiotic stress and exogenous ABA, subsequently, bZIP protein can also be activated and bind with ABA responsive element, thereby triggering the expression of downstream genes (Choi et al, ABFs, a family of ABA-responsive element binding factors. J Biol Chem. 2000 Jan. 21; 275(3):1723-30). In Arabidopsis and rice, several bZIP transcriptional factors involved in stress response, such as ABF1/2/3, ABI5 and OsbZIP23, etc, have been identified. Other factors related to stress response, e.g. CBF1/2/3 in Arabidopsis and SNAC1, DST1 in rice, have also been identified in other transcription factor families, such as DREB, NAC, Zinc finger, etc. Upon sensing upstream stress signals, these transcription factors directly bind to the promoter regions of their target genes and thereby control the adaptive ability of plants to stress.

During the identification process of stress responsive genes, it is found that some genes are directly involved in regulation of stress response, while some other genes do not participate stress response or do not exhibit their functions in natural conditions but do show their regulatory capability on stress response when being artificially modified. In plants, these genes will be subject to genetic modification depending on stress regulation during their effecting process or sometimes relevant modification or conformational change in the expression products encoded by these genes are required. For example, the full length form of Arabidopsis thaliana transcription factor DREB2A shows rather weak transcriptional activation activity, i.e. overexpression of DREB2A cannot remarkably induce the expression of downstream genes and stress resistance of overexpression plants has no significant change, while the construct with one NRD (negative regulatory domain) deleted from DREB2A exhibits strong transcriptional activation activity and overexpression of the construct remarkably increases the expression of downstream genes and enhances drought resistance of transgenic plants (Yoh Sakuma et al, Functional analysis of an Arabidopsis transcription factor, DREB2A, involved in drought-responsive gene expression. Plant Cell. 2006 May; 18(5):1292-309).

OsbZIP46 gene involved in the present invention is a member of rice bZIP family. The full length form of OsbZIP46 has no transcriptional activation activity and overexpression of the full length OsbZIP46 does not enhance drought resistance of transgenic plants.

### SUMMARY OF THE INVENTION

In the present invention, it has been found that there is a negative regulatory region in the OsbZIP46 protein. The present invention relates to a genetically modified OsbZIP46 gene, wherein the protein encoded by said gene lacks the negative regulatory region of the OsbZIP46 protein. Particularly, the negative regulatory region is domain D of the OsbZIP46 protein.

The present invention also relates to use of a genetically modified OsbZIP46 gene in genetically improving drought resistance of a plant. Therefore, the present invention also relates to a method of improving drought resistance of a plant, wherein said plant is subject to a treatment so that a genetically modified OsbZIP46 gene according to the present invention is expressed in said plant.

Additionally, the present invention relates to genetically modified plant or a cell thereof or transgenic plant or a cell thereof, wherein a genetically modified OsbZIP46 gene according to the present invention is comprised in the chromosome of the plant or plant cell so that it can be expressed, preferrably overexpressed, under drought conditions.

### DETAILED DESCRIPTION OF THE INVENTION

Unless specifically defined, all technical and scientific terms used herein have the same meaning as commonly understood by persons of ordinary skill in the art. The procedures for preparing and screening transgenic plants described below are well known and commonly employed by persons of ordinary skill in the art.

As mentioned above, the full length form of OsbZIP46 has no transcriptional activation activity and overexpression of the full length OsbZIP46 does not enhance drought resistance of transgenic plants. The inventors now have found that there is a negative regulatory region for the transcriptional activation activity in the OsbZIP46 protein. Once this negative regulatory region is deleted or functionally eliminated, the resultant genetically modified OsbZIP46 shall exhibit constitutive transcriptional activation activity. Overexpression of such genetically modified OsbZIP46 gene in a plant, especially in rice, shall confer increased drought resistance of the plant. The OsbZIP46 gene can originate from any suitable plant and preferably it is originate from rice.

Therefore, the present invention provides a genetically modified OsbZIP46 gene, wherein the protein encoded by the gene lacks a negative regulatory region for the transcriptional activation activity. Particularly, it has been further found that the negative regulatory region of OsbZIP46 is the domain D of OsbZIP46 protein. Domain D of the OsbZIP46 protein of rice is a region from about amino acid position 121 to about amino acid position 149 of the OsbZIP46 protein.

The term “lack” or “lacking” as used in herein in connection with a negative regulatory region or domain D of OsbZIP46 protein, it refers to deletion of the whole negative regulatory region or domain D or functional elimination of the negative region or domain D. “Functionally elimination” refers to losing or substantially losing the negative regulation function of the negative regulatory region or domain D so that the resultant modified OsbZIP46 protein exhibits increased transcriptional activation activity. Functional elimination can be achieved by partially deleting the negative regulatory region or domain D or one or more amino acids substitution or addition in the region or domain. Based on the inventors' finding of the negative region or domain D in the region of about aa 121 to about aa 149, one skilled in the art can easily find specific deletion/substitution/addition of amino acids in this region according to the teachings of the present invention and the knowledge of the art so that the negative regulation function of the region is eliminated or lost.

By deletion or functional elimination of the negative regulatory region or domain D of OsbZIP46 protein, the modified protein shall exhibits increases transcriptional activation activity. The genetically modified gene according to the present invention encodes a protein that exhibits constitutive transcriptional activation activity. Overexpression of the genetically modified OsbZIP46 gene of the present invention shall confer increased drought resistance of a plant, especially rice plants.

The term “increase” as used herein in connection with transcriptional activation activity or drought resistance of a plant, it refers to increase with statistical significance, e.g., about 10%, 20%, 30%, 40% 50%, 60%, 70%, 80%, 90%, 100%, 150%, 200%, 300%, 400%, 500% or even higher of increase.

In practice, deletion of the whole negative region or domain D may be achieved by deletion of a larger fragment comprising the region or domain. In a preferred embodiment, the genetically modified gene according to the present invention encodes an amino acid sequence as set forth in SEQ ID NO: 2. SEQ ID NO:2 is the amino acid sequence of a modified OsbZIP46 protein in which the region of aa 121 to aa 219 of the OsbZIP46 protein has been deleted. In a more preferable embodiment, the genetically modified gene according to the present invention comprises a nucleotide sequence as set forth in SEQ ID NO.1. The genetically modified OsbZIP46 gene represented by SEQ ID NO:1 or SEQ ID NO:2 is designated as OsbZIP46CA1 gene in the present invention.

The genetically modified OsbZIP46 gene of the present invention can be used in genetically improving drought resistance of a plant. The plant can be any plant that needs to be improved in drought resistance. For example, the plant is selected from the group consisting of corn, cotton, soybean, rice and wheat plants. Particularly, the present invention provides a method of improving drought resistance of a plant, wherein said plant is subject to a treatment so that a genetically modified OsbZIP46 gene according to the present invention is expressed in said plant. Preferably, the treatment is transformation of said plant by a recombinant DNA construct comprising said gene.

Again, the plant can be any plant such as a plant selected from the group consisting of corn, cotton, soybean, rice and wheat plants.

Therefore, the present invention also provides a recombinant DNA construct and preferably an expression vector loaded with a genetically modified OsbZIP46 gene according to the present invention. The expression vector loaded with a genetically modified OsbZIP46 gene according to the present invention can be introduced into plant cells with Ti plasmid and plant viral vector by the conventional biological technology methods such as direct transformation, microinjection and electroporation (Weissbach, 1998, Method for Plant Molecular Biology VIII, Academy Press, New York, pp. 411-463; Geiserson and Corey, 1998, Plant Molecular Biology (2nd Edition)).

The expression vector comprising the genetically modified OsbZIP46 gene according to the present invention can be transformed into hosts (multiple plants including rice) to breed plant varieties with drought resistance.

Thus the present invention provides a transgenic plant or a cell thereof comprising a genetically modified gene as defined according to the present invention transformed in its chromosome. For example, the transgenic plant can be corn, cotton, soybean, rice or wheat plants.

Thus, the present invention also provides a genetically modified plant or a cell thereof, wherein the OsbZIP46 gene in its chromosome has been modified so that the protein encoded by said gene lacks a negative regulatory region of the OsbZIP46 protein. Preferably, said negative regulatory region is domain D of the OsbZIP46 protein. The protein encoded by the modified gene exhibits constitutive transcriptional activation activity. For example, the modified gene encodes an amino acid sequence as set forth in SEQ ID NO: 2. More preferably, the genetically modified plant or a cell thereof according to the present invention has a modified OsbZIP46 gene with a nucleotide sequence as set forth in SEQ ID NO.1. The genetically modified plant or a cell thereof according to the present invention can be any plant such as a plant selected from the group consisting of corn, cotton, soybean, rice and wheat plants.

One object of the present invention relates to the use of OsbZIP46CA1, a novel form of OsbZIP46 (a member of bZIP transcriptional factor family) in controlling rice drought resistance improvement. The applicant cloned the fragment of rice bZIP transcriptional factor OsbZIP46 resulted from deletion of position 361 to position 657 base pairs (totally 297 base pairs, relating to the portion encoding 99 amino acids) and named the gene as OsbZIP46CA1 (OsbZIP46 Constitutive Active form). In the present invention, the applicant cloned and used a cDNA fragment containing OsbZIP46CA1 gene, which confers rice increased resistance to drought stress, wherein the nucleotide sequence containing OsbZIP46CA1 gene is set forth in SEQ ID NO:1 with 678 bp in length, wherein the corresponding amino acid sequence is set forth in SEQ ID NO:2 with 225 amino acids.

With genetic modification methods, the applicant cloned OsbZIP46CA1 fragment with internal 297 bp deleted from OsbZIP46 and the results show that OsbZIP46CA1 has a strong transcriptional activation activity and overexpression of OsbZIP46CA1 gene by transgenic technology enhances the drought resistance of rice. Therefore, the present invention has established a novel form of OsbZIP46 gene, OsbZIP46CA1, remarkably increasing rice drought resistance, which is of great importance in genetically improving rice drought resistance.

Particularly, the present invention provides:

Use of artificial modified OsbZIP46CA1 gene in genetically improving rice drought resistance, characterized in that the nucleotide sequence of said gene is set forth in SEQ ID NO: 1;

Use of artificial modified OsbZIP46CA1 gene in genetically improving rice drought resistance, characterized in that the amino acid sequence encoded by said gene is set forth in SEQ ID NO: 2; and

Use of artificial modified OsbZIP46CA1 gene in genetically improving rice drought resistance, characterized in that the protein encoded by said gene lacks negative regulatory region of OsbZIP46 protein and exhibits constitutive transcriptional activation activity.

A transgenic “plant cell” means a plant cell that is transformed with stably-integrated, non-natural, recombinant polynucleotides, e.g. by Agrobacterium-mediated transformation or by bombardment using microparticles coated with recombinant polynucleotides. A plant cell of this invention can be an originally-transformed plant cell that exists as a microorganism or as a progeny plant cell that is regenerated into differentiated tissue, e.g. into a transgenic plant with stably-integrated, non-natural recombinant polynucleotides in its chromosomal DNA, or seed or pollen derived from a progeny transgenic plant.

A “transgenic” plant or seed means one whose genome has been altered by the stable incorporation of recombinant polynucleotides in its chromosomal DNA, e.g. by transformation, by regeneration from a transformed plant from seed or propagule or by breeding with a transformed plant. Thus, transgenic plants include progeny plants of an original plant derived from a transformation process including progeny of breeding transgenic plants with wild type plants or other transgenic plants. The enhancement of a desired trait can be measured by comparing the trait property in a transgenic plant which has recombinant DNA conferring the trait to the trait level in a progenitor plant.

“Gene expression” means the function of a cell to transcribe recombinant DNA to mRNA and translate the mRNA to a protein. For expression the recombinant DNA usually includes regulatory elements including 5′ regulatory elements such as promoters, enhancers, and introns; other elements can include polyadenylation sites, transit peptide DNA, markers and other elements commonly used by those skilled in the art. Promoters can be modulated by proteins such as transcription factors and by intron or enhancer elements linked to the promoter.

“An increased level” of expression means an increase in the gene expression that is helpful for the drought resistance of the plant, e.g., at least about 5%, at least about 10%, at least about 15%, at least about 20%, at least about 25%, at least about 30%, at least about 40% at least about 50%, at least about 100%, at least about 200% increase in comparison with an identical control without the treatment of the present invention.

“Recombinant polynucleotide” means a DNA construct that is made by combination of two otherwise separated segments of DNA, e.g., by chemical synthesis or by the manipulation of isolated segments of nucleic acids by genetic engineering techniques. Recombinant DNA can include exogenous DNA or simply a manipulated native DNA. Recombinant DNA for expressing a protein in a plant is typically provided as an expression cassette which has a promoter that is active in plant cells operably linked to DNA encoding a protein, linked to a 3′ DNA element for providing a polyadenylation site and signal. Useful recombinant DNA also includes expression cassettes for expressing one or more proteins conferring stress tolerance.

Recombinant DNA constructs generally include a 3″ element that typically contains a polyadenylation signal and site. Well-known 3′ elements include those from Agrobacterium tumefaciens genes such as nos 3′, tml 3′, ttnr 3′, tms 3′, ocs 3\ tr73′, e.g., disclosed in U.S. Pat. No. 6,090,627. 3′ elements from plant genes such as a rice glutelin gene, a rice lactate dehydrogenase gene and a rice beta-tubulin gene are disclosed in U.S. published patent application 2002/0192813 A1.

The expression vector carrying the genetically modified OsbZIP46 gene of the present invention can be introduced into plant cells with Ti plasmid or plant viral vector using the conventional biological technology methods such as direct DNA transformation, microinjection and electroporation (Weissbach, 1998, Method for Plant Molecular Biology VIII, Academy Press, New York, pp. 411-463; Geiserson and Corey, 1998, Plant Molecular Biology (2nd Edition)).

The expression vectors comprising the genetically modified OsbZIP46 gene of the present invention can be transformed into multiple host plants including rice to breed plant varieties with drought resistance.

### Plant Cell Transformation Methods

Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat) and Agrobacterium-mediated transformation is described in U.S. Pat. No. 5,159,135 (cotton); U.S. Pat. No. 5,824,877 (soybean); U.S. Pat. No. 5,591,616 (corn); and U.S. Pat. No. 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, for example to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plants line for selection of plants having an enhanced trait. In addition to direct transformation of a plant with a recombinant DNA, transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example drought resistance or pest resistance, to produce progeny plants having recombinant DNA that confers both traits.

In the practice of transformation DNA is typically introduced into only a small percentage of target plant cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptl1), hygromycin B (aph IV) and gentamycin (aac3 and aacCA) or resistance to herbicides such as glufosinate {bar or pat) and glyphosate (aroA or EPSPS). Examples of such selectable are illustrated in U.S. Pat. Nos. 5,550,318; 5,633,435; 5,780,708 and 6,118,047. Selectable markers which provide an ability to visually identify transformants can also be employed, for example, a gene expressing a colored or fluorescent protein such as a luciferase or green fluorescent protein (GFP) or a gene expressing a fteto-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO2, and 25-250 microeinsteins m⁻¹s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from the plant cells of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) including the recombinant DNA, for example multiple plants from 2 to 20 or more transgenic events. Transgenic plants grown from transgenic seed provided herein demonstrate improved agronomic traits that contribute to increased yield or enhanced water deficit tolerance or both.

Not all transgenic events will be in transgenic plant cells that provide plants and seeds with an enhanced or desired trait depending on factors, such as location and integrity of the recombinant DNA, copy number, unintended insertion of other DNA, etc. As a result transgenic plant cells of this invention are identified by screening transformed progeny plants for enhanced water deficit stress tolerance and yield. For efficiency a screening program is designed to evaluate multiple transgenic plants preferably with a single copy of the recombinant DNA from 2 or more transgenic events.

Since the expression of the gene of the present invention is to be induced by drought stress, the gene of the present invention can be inserted into suitable expression vector with combination with any drought inducible promoter of interest and transformed into plant hosts, wherein the gene expression thereby can be induced by drought condition, enhancing the drought resistance of the plant thereof.

Further illustration of the present invention is given below in reference to the figures and examples.

### BRIEF DESCRIPTION OF THE DRAWINGS

SEQ ID NO: 1 is the nucleotide acid sequence containing the coding region of the OsbZIP46CA1 gene constructed and cloned in the present invention with 678 bp in length, and the amino acid sequence of the corresponding protein thereof is set forth in SEQ ID NO: 2 with 225 amino acids in length.

FIG. 1. Transcriptional activation assay of OsbZIP46CA1 protein. Sequence analysis shows that there are four conservative domains (a-d) in the transcriptional regulatory region of the transcription factor OsbZIP46, wherein the proteins lacking domain d (from aa121 to aa149) (dC5, dC6, CA1) have strong activity of transcriptional activation.

FIG. 2. The schematic diagram of OsbZIP46CA1 overexpression vector and expression level of transgenic overexpression plant. WT is wild type control.

FIG. 3. Phenotype of OsbZIP46CA1 overexpression plant under drought stress. OX1 and OX7 are overexpression lines and ZH11 is wild type plant.

FIG. 4. Survival rates of OsbZIP46CA1 overexpression plants under drought stress. OX1 and OX7 are overexpression lines and ZH11 is wild type plant.

FIG. 5. Dehydration rates in the leaves cut of OsbZIP46CA1 overexpression plants. CA1U-1 OX and CA1U-3 OX are overexpression lines, CA1U-4 NOX is negative transgenic line and ZH11 is wild type plant.

FIG. 6. Phenotype of OsbZIP46CA1 overexpression plants under osmosis stress. OsbZIP46CA1U-1 and OsbZIP46CA1U-5 are OsbZIP46CA1 overexpression lines, OsbZIP46U-15 is fall length OsbZIP46 overexpression line (as control) and ZH11 is wild type control.

FIG. 7. Height statistics of OsbZIP46CA1 overexpression plants under osmosis stress which is corresponding to the data in FIG. 6.

FIG. 8. The induced expression patterns of downstream genes in OsbZIP46CA1 overexpression plant. CA1OX-2 and CA1OX-5 are OsbZIP46CA1 overexpression lines, FLOX-9 and FLOX-15 are full length OsbZIP46 overexpression lines (as control) and ZH11 is wild type control.

The following examples are included to demonstrate embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention, therefore all matter set forth or shown in the accompanying drawings and examples is to be interpreted as illustrative and not in a limiting sense.

### EXAMPLES

The following examples describe methods for isolating and cloning the DNA fragment comprising the entire encoding region of OsbZIP46CA1 gene and for verifying the function of OsbZIP46CA1 gene.

### Example 1

### Transcriptional Activation Assay of OsbZIP46CA1 Protein

The inventers performed transcriptional activation assay of OsbZIP46CA1 protein in yeast using ProQuest Two-Hybrid System (Invitrogen, Carlsbad, Calif., USA).

The full length construct and a series of deletion mutants of OsbZIP46 gene were obtained using PCR. By searching in two databases, the Annotation No. of OsbZIP46 gene is LOC_Os06g10880 and AK103188 in Rice Genome Annotation Project-TIGR (http://rice.plantbiology.msu.edu/) and KOME (http://cdna01.dna.affrc.go.jp/cDNA/) respectively. A cDNA clone (Accession Number: BI103-O13) comprising partial sequence of 5′ coding region of OsbZIP46 gene was identified by searching in Minghui63 normalized cDNA Library of all development stages (Chu et al, Construction and characterization of normalized whole-life-cycle cDNA library of rice. Chinese Science Bulletin, 2003, 48: 229-235) constructed by Key Laboratory of Crop Genetic Improvement, Huazhong Agricultural University (public website of the library is http://redb.ncpgr.cn/modules/redbtools/). The clone was picked up from the library and the plasmid was extracted. Sequencing was conducted from the two ends with universal primers (5′-ATTTAGGTGACACTATA-3′) and T7 (5′-TAATACGACTCACTATAGGG-3′) using ABI7500 instrument in our laboratory. Then the complete open reading frame of OsbZIP46 gene in clone BI103-O13 was confirmed by sequence aligning with full length cDNA of OsbZIP46 gene in KOME database. PCR amplification was carried out using the above clone as template with forward primer OsbZIP46p32F1-attB1 (5′-ggacaagtttgtacaaaaaagcaggctTGGAGTTGCCGGCGGATG-3′) and the following reverse primers: OsbZIP46p32R1-attB2 (5′-ggaccactttgtacaagaaagctgggtTCAGCATGGACCAGTCAGTG-3′), OsbZIP46p32R2-attB2 (5′-ggaccactttgtacaagaaagctgggtTCAAGTGATTCTCTCCATGAC-3′), OsbZIP46p32R4-attB2 (5′-ggaccactttgtacaagaaagctgggtTCACTCGACGGTAGGGCCCTTC-3′), OsbZIP46p32R5-attB2 (5′-ggaccactttgtacaagaaagctgggtTCACAGCATCCCGTTGGCGAGC-3′), OsbZIP46CA0R-attB2 (5′-ggaccactttgtacaagaaagctgggtTCACATGTCCTCCCGCACGAC-3′), OsbZIP46p32R6-attB2 (5-ggaccactttgtacaagaaagctgggtTCACGCAGCCGCCGCCGCCGCGGGGTC-3′), OsbZIP46p32R7-attB2 (5′-ggaccactttgtacaagaaagctgggtTCACGCCGCCGGCGCTATGGCCTG-3′) and the conditions were set as: predenaturation for 5 min at 94° C.; 94° C. 30 sec, 55° C. 30 sec, 72° C. 1 min for 32 cycles; extension for 5 min at 72° C. The fragments were named as FL, dC1, dC3, dC4, dC4.5, dC5 and dC6 respectively, representing full length OsbZIP46 and the fragments with 19, 85, 144, 175, 204 and 264 amino acids deleted at 3′ terminus of OsbZIP46. OsbZIP46CA1 clone was amplified by using staggered extension PCR method. Firstly, a PCR amplification was conducted with the above clone BI103-O13 as template with two pairs of primers OsbZIP46p32F1-attB1 (5′-ggacaagtttgtacaaaaaagcaggctTGGAGTTGCCGGCGGATG-3′)/OsbZIP46CuActR1 (5-CGCAGCCGCCGCCGCCGCGGGGTC-3′) and OsbZIP46CuActF1 (5′-GACCCCGCGGCGGCGGCGGCTGCGTCGCCGGTGCCTTACCCA-3′)/OsbZIP46p32R1-attB2 (5′-ggaccactttgtacaagaaagctgggtTCAGCATGGACCAGTCAGTG-3′) and the conditions were set as: predenaturation for 5 min at 94° C.; 94° C. 30 sec, 55° C. 30 sec, 72° C. 1 min for 32 cycles; extension for 5 min at 72° C. The second round of PCR amplification was carried out using the mixture of two products of the PCR reaction above as template with primer pair OsbZIP46p32F1-attB1 (5′-ggacaagthgtacaaaaaagcaggctTGGAGTTGCCGGCGGATG-3′)/OsbZIP46p32R1-attB2 (5′-ggaccactttgtacaagaaagctgggtTCAGCATGGACCAGTCAGTG-3′) and the conditions were set as: predenaturation for 5 min at 94° C.; 94° C. 30 sec, 55° C. 30 sec, 72° C. 1 min for 35 cycles; extension for 5 min at 72° C., wherein the primers were added in the 8^{th} cycle and the PCR amplified fragment was named as OsbZIP46CA1, namely the fragment with position 361 to 657 base pairs deleted (corresponding to position 121 to 219 amino acids).

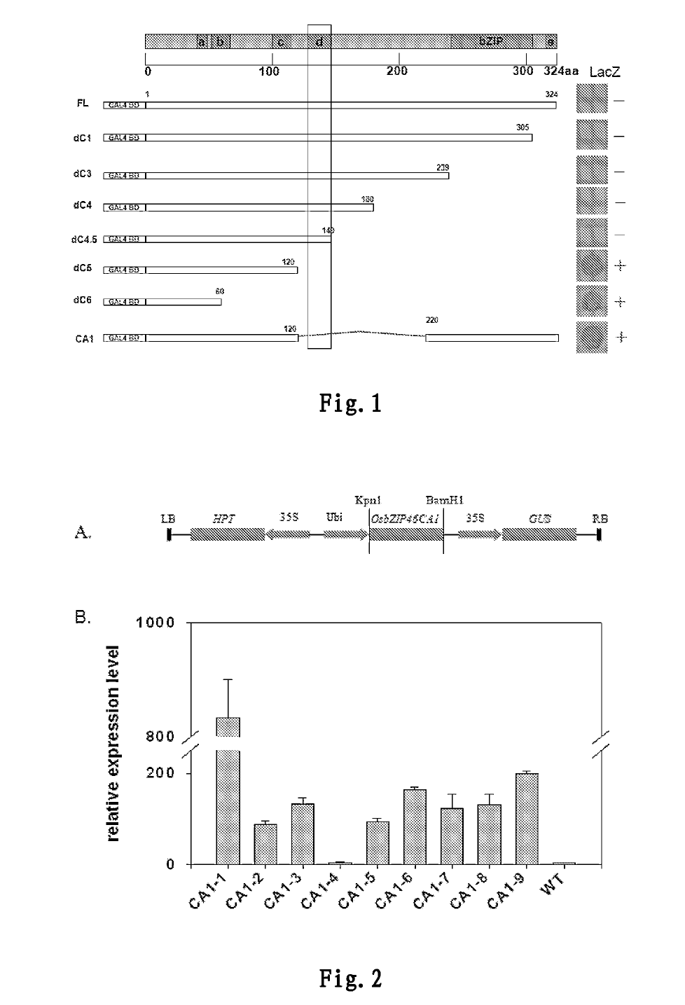

Subsequently, transcriptional activity analysis was carried out by cloning these fragments into corresponding vectors and transforming them into yeast. Particularly, the amplified fragments were recombined into intermediate vector pDONR221 (Purchased from Invitrogen Co.) using BP reaction, wherein the resulting clones were sequenced for verification and designated as FL-p221/dC1-p221/dC3-p221/dC4-p221/dC4.5-p221/dC5-p221/dC6-p221/OsbZIP46 CA1-p221. The relevant fragments were then recombined into the yeast GAL4-DB fusion expression vector pDONR221 (Purchased from Invitrogen Co.) by LR reaction, wherein the resultant clones were designated as FL-p32/dC1-p32/dC3-p32/dC4-p32/dC4.5-p32/dC5-p32/dC6-p32/OsbZIP46CA1-p32. Then they were transformed into yeast strain May 203 (Purchased from Invitrogen Co., genotype: MATα, leu2-3, 112, trp1-901 his3Δ200, ade2-101, gal4Δ, gal80Δ, SPAL10::URA3, GAL1::lacZ,HIS3UAS GAL1::HIS3@LYS2, can1R, cyh2R). The activity of β-Galactosidase was determined in combination with Colony-lift filter by measuring whether yeast colony exhibited blue color for determining the expression of the reporting gene LacZ, thereby to determine whether said gene has transcriptional activation activity (for more information, see manuals from Invitrogen Co.) (FIG. 1). The results demonstrated that full length and the fragments with 19-, 85-, 144-, 175-amino acid deletions at 3′ terminal of OsbZIP46 protein had no transcriptional activation activity, while the fragments with 204-amino acid deletion (dC6) 3′ terminal, 264-amino acid deletion (dC7) at 3′ terminal or deletion of position 121-219 amino acids (OsbZIP46CA1) exhibited strong transcriptional activation activity, suggesting that a fragment negatively regulating the transcriptional activation activity of OsbZIP46 is present between position 120 amino acid and position 149 amino acid of OsbZIP46. Since OsbZIP46CA1 is a novel artificially modified form of OsbZIP46 with negative regulatory region deleted and exhibiting strong transcriptional activation activity and also comprises bZIP (basic Leucine Zipper) domain indispensable for DNA binding and oligomerization, OsbZIP46CA1, as a constitutive active form of OsbZIP46, is the main subject of the present invention (FIG. 1).

### Example 2

### Construction and Genetic Transformation of the Overexpression Vector of OsbZIP46CA1 Gene

Construction of OsbZIP46CA1 Expression Vector:

In order to verify the stress resistant function of OsbZIP46CA1 gene, it was overexpressed in rice and the function was investigated by verifying the phenotype of transgenic plants thereof. OsbZIP46CA1 expression vector was constructed as follows. With the obtained clone OsbZIP46CA1-p32 comprising OsbZIP46CA1 as template, PCR amplification was conducted using primers OsbZIP46-F-Kpn1(5′-ATAggtaccATGGAGTTGCCGGCGGATG-3′)/OsbZIP46-R-BamH1 (5′-ATAggatccTCAGCATGGACCAGTCAGTG). Then, the PCR product was enzymatically cleaved with KpnI and BamHI; meanwhile, the genetic transformation vector pCAMBIA1301U with the ubiquitin promoter was enzymatically cleaved in the same way (pCAMBIA1301U was reconstructed based on genetic transformation vector pCAMBIA1301 commonly used internationally, an Agrobacterium mediated vegetable genetic transformation vector carrying corn ubiquitin promoter with constitutive and over expression characteristics). After cleavage, the products of enzymatical cleavage were extracted and purified with chloroform: iso-pentanol (24:1 v/v). Linkage reaction between the digested OsbZIP46CA1 fragment and digested pCAMBIA1301U vector was conducted and then the reaction mixture was used to transform. E. coli DH10β (the E. coli DH10β strain was purchased from Promega Co.). By screening positive clones with enzymatical digestion, the recombinant plasmid obtained was designated as OsbZIP46CA1-1301U (FIG. 2A).

The Genetic Transformation Protocol:

By using the Agrobacterium mediated rice genetic transformation system (described as follows), the above overexpression vector OsbZIP46CA1-1301U was introduced into the rice variety “Zhonghua 11” and transgenic plants were then obtained by precultivation, infestation, co-culture, screening for callus with hygromycin resistance, differentiation, rooting, seedling training and transplanting. The above mentioned Agrobacterium mediated rice (Zhonghua 11) genetic transformation method (system) has optimized based on the method reported by Hiei, et al (Efficient transformation of rice, Oryza sativa L., mediated by Agrobacterium and sequence analysis of the boundaries of the T-DNA, Plant J, 6:271-282, 1994).

The genetic transformation protocol of the present example is as follows.

(1) Electrotransformation: Electrotransform the plasmid OsbZIP46CA1-1301U overexpressing target gene into Agrobacterium strain EHA105 under 1800V, plate the electrotransformation mixture on LA medium and then screen the positive clones to be used in the following callus transformation.

(2) Callus Induction: Mature rice seeds of ZHONGHUA 11 were deshelled, then treated with 70% alcohol for 1 minute and disinfected on the surface of the seeds with 0.15% HgCl₂ for 15 minutes. The seeds were washed with sterilized water for 4-5 times. The sterilized seeds were put on the induction medium (see below for components); The inoculated callus induction medium was placed in darkness and cultured for 4 weeks at 25±1° C.

(3) Callus Subculture: The bright yellow, compact and relatively dry embryogenic callus was selected, put onto the subculture medium (see below for components), and cultured in darkness for 2 weeks at 25±1° C.

(4) Pre-Culture: The compact and relatively dry embryogenic callus was selected, put onto the pre-culture medium (see below for components), and cultured in darkness for 2 weeks at 25±1° C.

(5) Agrobacterium Culture: Agrobacterium EHA105 (from CAMBIA, commercial strain, carrying the overexpression vector OsbZIP46CA1-1301U of the present invention) was precultured on the selection LA medium (see below for components) at the temperature of 28° C. for 2 days. Said Agrobacterium was transferred into suspension medium (as described below) in swing bed at 28° C. for 2-3 hours.

(6) Agrobacterium Infection: The pre-cultured calluses were transferred into a sterilized bottle. The Agrobacterium suspension was adjusted to OD₆₀₀ 0.8-1.0. The calluses were immersed in the Agrobacterium suspension for 30 minute; The calluses were transferred on sterilized filter paper and dried, and then cultured onto the cocultivation medium (see below for components) for 3 days at 19-20° C.

(7) Washing and Selection Culture of Callus: The calluses were washed with sterilized water until no agrobacrium was observed; immersed in sterilized water containing 400 ppm carbenicillin (CN) for 30 minutes; transferred on sterilized filter paper and dried; and transferred on the selection medium (see below for components) and cultured for selection for 2-3 times, 2 weeks for each time (The carbenicillin concentration was 400 ppm for the first culture, and 250 ppm for the second and subsequent cultures. The hygromycin concentration was 250 ppm.)

(8) Differentiation: The resistant calluses were transferred to the pre-differentiation medium (see below for components) and cultured in darkness for 5-7 weeks. The pre-differentiated calluses were then transferred to differentiation culture medium (see below for components) and cultured in lighting (3500 lux) at 26° C.

(9) Rooting: The roots generated during the differentiation were cut off. Then the plants were transferred to the rooting culture medium and cultured in lighting at 26° C. for 2-3 weeks.

(10) Transplantation: The residual medium on roots of the plants was washed off, the seedlings with well-grown roots were transplanted into greenhouse, and moisture was maintained in the first several days.

The Components and Formulas of the Mediums:

(1) The Abbreviations of the Reagents and Solutions

The abbreviations of the plant hormones used in the mediums of the present invention are: 6-BA (6-Benzyladenine); CN (Carbenicillin); KT (Kinetin); NAA (Napthalene acetic acid); IAA (Indole-3-acetic acid); 2,4-D (2,4-Dichlorophenoxyacetic acid); AS (Acetosringone); CH (Casein Enzymatic Hydrolysate); HN (Hygromycin B); DMSO (Dimethyl Sulfoxide); N6max (N6 solution with major elements); N6mix (N6 solution with trace elements); MSmax (MS solution with major elements); MSmix (MS solution with trace elements).

(2) The Formula of the Main Solutions

1) Preparation of Concentrated Solution of N6 Medium with Major Elements (10× Concentrated Solution):

| | |
|---|---|
| Potassium nitrate (KNO3) | 28.3 g |
| Monopotassium phosphate (KH2PO4) | 4.0 g |
| Ammonium sulfate ((NH4)2SO4) | 4.63 g |
| Magnesium sulfate (MgSO4•7H2O) | 1.85 g |
| Calcium chloride (CaCl2•2H2O) | 1.66 g |

Dissolve them one by one and add water to the final volume 1000 ml at room temperature.

2) Preparation of Concentrated Solution of N6 Medium with Trace Salts (10× Concentrated Solution):

| | |
|---|---|
| Potassium iodide (KI) | 0.08 g |
| Boracic acid (H₃BO₃) | 0.16 g |
| Manganese sulfate (MnSO₄•4H₂O) | 0.44 g |
| Zinc sulfate (ZnSO₄•7H₂O) | 0.15 g |

Add water to the final volume 1000 ml at room temperature.

3) Preparation of the Ferric Salt (Fe₂EDTA) Stock Solution (100×)

800 ml of double distilled water was heated to 70° C., 3.73 g of Disodium Ethylene Diamine Tetraacetic Acid (Na2EDTA.2H2O) was added therein. After dissolving completely, the solution was kept in 70° C. water bath for 2 hours and water was added to a final volume 1000 ml and then the solution was kept at 4° C. for use.

4) Preparation of the Vitamin Stock Solution (100×)

| | |
|---|---|
| Nicotinic acid | 0.1 g |
| Vitamin B1 (Thiamine HCl) | 0.1 g |
| Vitamin B6 (Pyridoxine HCl) | 0.1 g |
| Glycine | 0.2 g |
| Inositol | 10 g |

Add water to a final volume 1000 ml and then keep the solution at 4° C. for use.

5) Preparation of the Concentrated Solution of MS Medium with Major Elements (10×):

| | |
|---|---|
| Ammonium nitrate (NH₄NO₃) | 16.5 g |
| Potassium nitrate | 19.0 g |
| Monopotassium phosphate | 1.7 g |
| Magnesium sulfate | 3.7 g |
| Calcium chloride | 4.4 g |

Dissolve the components and add water to the final volume 1000 ml at room temperature.

6) Preparation of the Concentrated Solution of MS Medium with Trace Elements (10×)

| | |
|---|---|
| Potassium iodide | 0.083 g |
| Boracic acid | 0.62 g |
| Manganese sulfate | 0.86 g |
| Sodium Molybdate (Na₂MoO₄•2H₂O) | 0.025 g |
| Copper sulphate (CuSO₄•5H₂O) | 0.0025 g |

Dissolve the components and add water to the final volume 1000 ml at room temperature.

7) Preparation of the 2,4-D Stock Solution (1 mg/ml)

100 mg 2,4-D was dissolved in 1 ml 1 N potassium hydroxide for 5 min, then 10 ml distilled water was added to completely dissolve followed by adding water to a final volume 100 ml and keeping the solution at room temperature.

8) Preparation of the 6-BA Stock Solution (1 mg/ml)

100 mg 6-BA was dissolved in 1 ml 1 N potassium hydroxide for 5 min, then 10 ml distilled water was added to dissolve completely followed by adding water to a final volume 100 ml and keeping the solution at room temperature.

9) Preparation of the Naphthylacetic Acid (NAA) Stock Solution (1 mg/ml):

100 mg NAA was dissolved in 1 ml 1 N potassium hydroxide for 5 min, then 10 ml distilled water was added to dissolve completely followed by adding water to a final volume 100 ml and keeping the solution at 4° C. for use.

10) Preparation of the Indoleacetic Acid (IAA) Stock Solution (1 mg/ml):

100 mg IAA was dissolved in 1 ml 1 N potassium hydroxide for 5 min, then 10 ml distilled water was added to dissolve completely followed by adding water to a final volume 100 ml and keeping the solution at 4° C. for use. Add 300 ml distilled water and 2.78 g ferric sulfate (FeSO₄.7H₂O) 2.78 g into a large triangular flask. Add 300 ml distilled water into another large triangular flask.

11) Preparation of the Glucose Stock Solution (0.5 g/ml):

125 g glucose was dissolved in distilled water by adding water to a final volume 250 ml followed by sterilization and storing the solution at 4° C.

12) Preparation of the AS Stock Solution:

0.392 g AS was dissolved completely in 10 ml DMSO. The solution was distributed into 1.5 ml centrifuge tubes and stored at 4° C. for use.

13) 1N Potassium Hydroxide Stock Solution

5.6 g potassium hydroxide was dissolved in distilled water by adding water to a final volume 100 ml and the solution was stored at 4° C. for use.

(3) The Formulas of the Mediums for Rice Genetic Transformation

1) Induction Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 2,4-D stock solution | 2.5 | ml |
| Proline | 0.3 | g |
| CH | 0.6 | g |
| Sucrose | 30 | g |
| Phytagel | 3 | g |

Add distilled water to 900 ml, adjust pH to 5.9 with 1N potassium hydroxide, boil the solution, add water to a final volume 1000 ml and distribute the solution into 50 ml-triangular flasks (25 ml/flask) followed by sealing and sterilization.

2) Subculture Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 2,4-D stock solution | 2.0 | ml |
| Proline | 0.5 | g |
| CH | 0.6 | g |
| Sucrose | 30 | g |
| Phytagel | 3 | g |

Add distilled water to 900 ml, adjust pH to 5.9 with 1 N potassium hydroxide, boil the solution, add water to a final volume 1000 ml and distribute the solution into 50 ml-triangular flasks (25 ml/flask) followed by sealing and sterilization.

3) Preculture Medium

| | | |
|---|---|---|
| N6max concentrated solution (10X) | 12.5 | ml |
| N6mix concentrated solution (100X) | 1.25 | ml |
| Fe²⁺EDTA stock solution (100X) | 2.5 | ml |
| Vitamin stock solution (100X) | 2.5 | ml |
| 2,4-D stock solution | 0.75 | ml |
| CH | 0.15 | g |
| Sucrose | 5 | g |
| Agar powder | 1.75 | g |

Add distilled water to 250 ml, adjust pH to 5.6 with 1 N potassium hydroxide followed by sealing and sterilization. Prior to use, heat and dissolve the medium, add 5 ml glucose stock solution and 250 μl AS stock solution and distribute the solution into dishes (25 ml/dish).

4) Coculture Medium

| | | |
|---|---|---|
| N6max concentrated solution (10X) | 12.5 | ml |
| N6mix concentrated solution (100X) | 1.25 | ml |
| Fe²⁺EDTA stock solution (100X) | 2.5 | ml |
| Vitamin stock solution (100X) | 2.5 | ml |
| 2,4-D stock solution | 0.75 | ml |
| CH | 0.2 | g |
| Sucrose | 5 | g |
| Agar powder | 1.75 | g |

Add distilled water to 250 ml, adjust pH to 5.6 with 1 N potassium hydroxide followed by sealing and sterilization. Prior to use, heat and dissolve the medium, add 5 ml glucose stock solution and 250 μl AS stock solution and distribute the solution into dishes (25 ml/dish).

5) Suspension Culture Medium

| | | |
|---|---|---|
| N6max concentrated solution (10X) | 5 | ml |
| N6mix concentrated solution (100X) | 0.5 | ml |
| Fe²⁺EDTA stock solution (100X) | 0.5 | ml |
| Vitamin stock solution (100X) | 1 | ml |
| 2,4-D stock solution | 0.2 | ml |
| CH | 0.08 | g |
| Sucrose | 2 | g |

Add distilled water to 100 ml, adjust pH to 5.4 and distribute the solution into two 100 ml-triangular flasks followed by sealing and sterilization. Prior to use, add 1 ml glucose stock solution and 100 μl AS stock solution.

6) Selection Culture Medium

| | | |
|---|---|---|
| N6max concentrated solution (10X) | 25 | ml |
| N6mix concentrated solution (100X) | 2.5 | ml |
| Fe²⁺EDTA stock solution (100X) | 2.5 | ml |
| Vitamin stock solution (100X) | 2.5 | ml |
| 2,4-D stock solution | 0.625 | ml |
| CH | 0.15 | g |
| Sucrose | 7.5 | g |
| Agar powder | 1.75 | g |

Add distilled water to 250 ml and adjust pH to 6.0 followed by sealing and sterilization. Prior to use, dissolve the medium, add 250 μl HN and 400 ppm CN and distribute the medium into dishes (25 ml/dish).

7) Pre-Differentiation Medium

| | | |
|---|---|---|
| N6max concentrated solution (10X) | 25 | ml |
| N6mix concentrated solution (100X) | 2.5 | ml |
| Fe²⁺EDTA stock solution (100X) | 2.5 | ml |
| Vitamin stock solution (100X) | 2.5 | ml |
| 6-BA stock solution | 0.5 | ml |
| KT stock solution | 0.5 | ml |
| NAA stock solution | 50 | μl |
| IAA stock solution | 50 | μl |
| CH | 0.15 | g |
| Sucrose | 7.5 | g |
| Agar powder | 1.75 | g |

Add distilled water to 250 ml, adjust pH to 5.9 with 1N potassium hydroxide followed by sealing and sterilization. Prior to use, dissolve the medium, add 250 μl HN and 200 ppm CN and distribute the medium into dishes (25 ml/dish).

8) Differentiation Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 6-BA stock solution | 2 | ml |
| KT stock solution | 2 | ml |
| NAA stock solution | 0.2 | ml |
| IAA stock solution | 0.2 | ml |
| CH | 1 | g |
| Sucrose | 30 | g |
| Phytagel | 3 | g |

Add distilled water to 900 ml, adjust pH to 6.0 with 1 N potassium hydroxide, boil the solution, add water to a final volume 1000 ml and distribute the solution into 50 ml-triangular flasks (50 ml/flask) followed by sealing and sterilization.

9) Rooting Medium

| | | |
|---|---|---|
| MSmax concentrated solution (10X) | 50 | ml |
| MSmix concentrated solution (100X) | 5 | ml |
| Fe²⁺EDTA stock solution (100X) | 5 | ml |
| Vitamin stock solution (100X) | 5 | ml |
| Sucrose | 30 | g |
| Phytagel | 3 | g |

Add distilled water to 900 ml, adjust pH to 5.8 with 1 N potassium hydroxide, boil the solution, add water to a final volume 1000 ml and distribute the medium into rooting tubes (25 ml/flask) followed by sealing and sterilization.

### Example 3

### Identification of the OsbZIP46CA1 Expression Level of OsbZIP46CA1 Overexpression Plants

In the present invention, the expression of OsbZIP46CA1 gene in the transgenic rice plants obtained in the above Example 2 was detected by fluorescent real time quantitative PCR method. Total RNAs were extracted using TRIZOL reagent (from Invitrogen Co.) according to the specification of the manufacturer and reverse transcribed to cDNA using reverse transcriptase SSIII (from Invitrogen Co.) according to the specification of the manufacturer. The reaction was conducted as follows: 65° C. 5 min, 50° C. 60 min, 70° C. 10 min. With the above reverse transcribed cDNA as template, OsbZIP46CA1 gene was specifically PCR amplified using primers OsbZIP46rtNTterminalF:5′-AAGCGCCGAGAAGGATTTC-3′ and OsbZIP46rtNTterminalR:5′-CCGCCGTCCAGATGTTG-3′. Meanwhile, a 76 bp fragment of the rice Actin1 gene (Accession No. X16280) was specifically amplified with primers (actin76F:5′-TGGCATCTCTCAGCACATTCC-3′ and actin76R:5′-TGCACAAT GGATGGGTCAGA-3′) as internal control for quantitative analysis. PCR reaction was conducted as follows: 95° C. 10 sec; 95° C. 5 sec, 60° C. 34 sec, 45 cycles. Fluorescent real time quantitative analysis was conducted during the reaction process. The results showed that the expression amount of OsbZIP46CA1 gene in most transgenic plants was significantly enhanced relative to that in wild type (FIG. 2B).

### Example 4

### Identification of the Drought Stress Phenotype of OsbZIP46CA1 Overexpression Plants

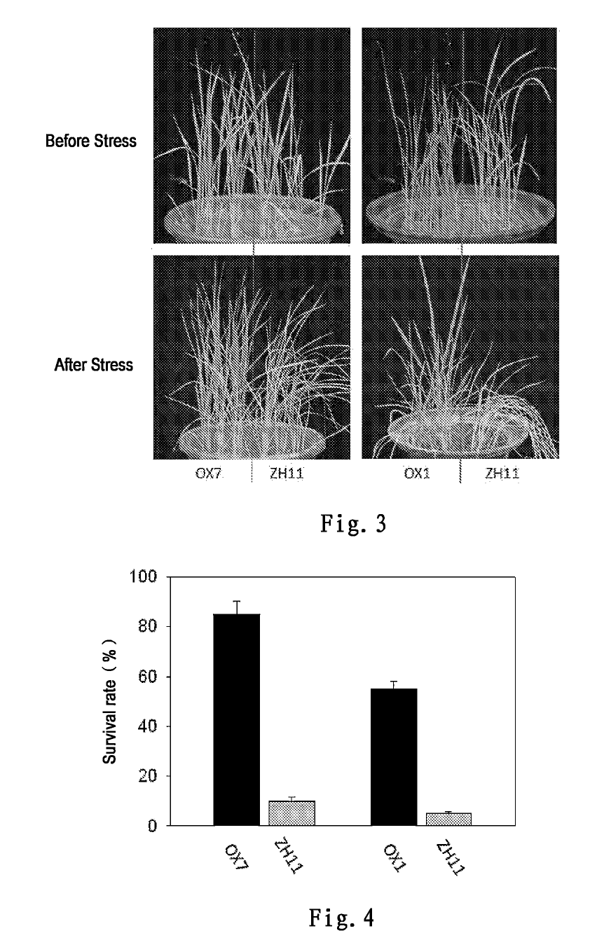

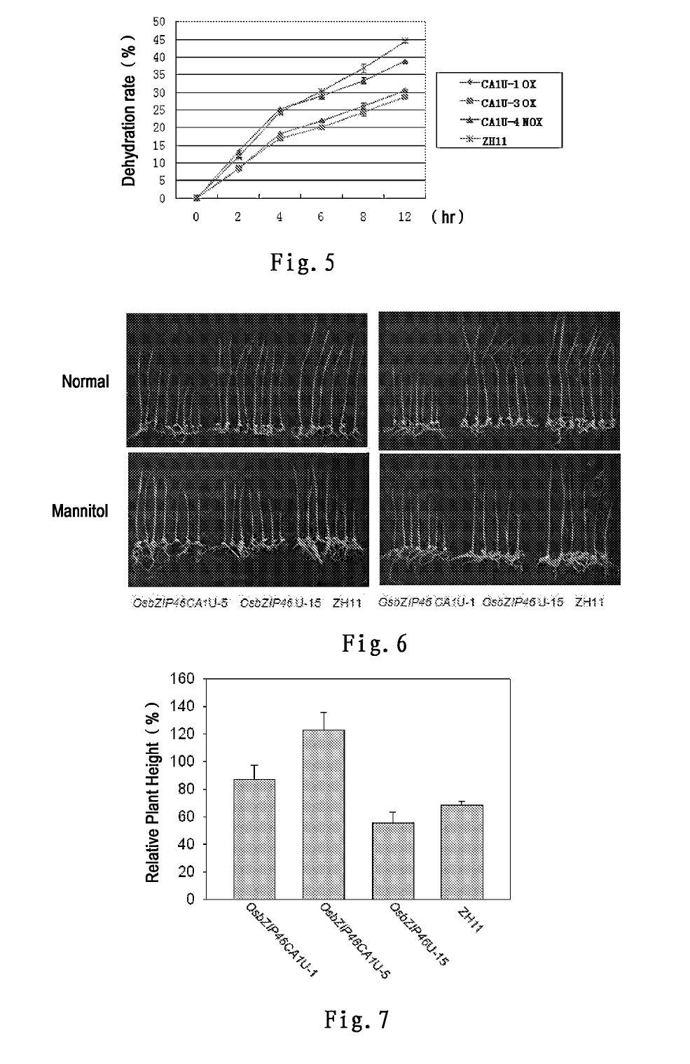

To verify the drought resistance of OsbZIP46CA1 overexpression plants, the drought resistant phenotype of OsbZIP46CA1 overexpression plants was identified. The seeds of two overexpression lines (OX7,OX1) and wild type line(ZH11) were deshelled and then sterilized (treated with 70% alcohol for 1 minute, disinfected with 0.15% HgCl₂ for 10 minutes and washed with sterilized water for several times). The seeds were germinated in 1/2 MS medium in the presence of 100 mg/L hygromycin and Zhonghua 11 (ZH11) lines were seeded in 1/2 MS medium absent of hygromycin one day later. 4-5 days later, well germinating seeds with consistent germinating rate were picked and planted into small round buckets with 25 transgenic plants and 25 wild type plants in each bucket. The soil used in the experiments was a mixture of soil and sands in a ratio of 2:3, the same amount of homologous soil with the same volume of water was added in each bucket and the water naturally leaked out to ensure the consistency of soil compactness. The experiment was in triplet. The plants in each bucket were stopped water supply at 3 to 4 leaves stage, until leaves completely rolled and leaf apex became white (generally 6 to 10 days, depending on the particular weather), and then rewatered for 5-7 days followed by observing the phenotype and survival rate of the plants. The results showed that during the process of drought stress, the leaves of wild type control plants rolled and showed chlorosis with increased wilting rate compared with overexpression plants. After a period of rewatering and recovery, almost all wild type control plants withered, while most of the plants in overexpression line were still alive and soon recovered with fresh green leaves. The further survival statistics shows that the survival rate in wild type control is lower than 10%, while the survival rate of overexpression plants remarkably higher than that of wild type plants in same bucket (more than 50%). The overexpression plants exhibited stronger resistance to drought stress compared with wild type control, showing that overexpression of OsbZIP46CA1 enhances drought resistance of plants (FIG. 3 and FIG. 4).

### Example 5

### Determination of Dehydration Rate of Ex Vivo Leaves of OsbZIP46CA1 Overexpression Plants

To further verify the drought resistance of OsbZIP46CA1 overexpression plants, the dehydration rates of leaves of OsbZIP46CA1 overexpression plants were determined. Partial leaves of plants at tillering stage were cut off, placed at room temperature and weighed at different time points followed by measuring dehydration rates according to the following formula: dehydration rate y(%)=(X0−Xn)/X0×100, wherein X0 is the starting weight of a plant and Xn is the weight of the plant at certain time point. The results showed that the dehydration rates in the leaves cut of OsbZIP46CA1 overexpression plants (CA1U-1 OX, CA1U-3 OX) were remarkably lower than those of transgenic negative control (CA1U-4 NOX) and wild type control (ZH11).

### Example 6

### Phenotype of the OsbZIP46CA1 Overexpression Plants Under Osmosis Stress

The seeds of two overexpression lines (OsbZIP46CA1U-1, OsbZIP46CA1U-5), wild type line (ZH11) and a full length OsbZIP46 overexpression line (OsbZIP46U-15) were deshelled and then sterilized (treated with 70% alcohol for 1 minute, disinfected with 0.15% HgCl₂ for 10 minutes and washed with sterilized water for several times). The seeds were germinated in 1/2MS medium in the presence of 100 mg/L hygromycin and Zhonghua 11 (ZH11) lines were seeded in 1/2 MS medium absent of hygromycin one day later. 2-3 days later, well germinating seeds with consistent germinating rate were picked and transferred into 1/2 MS medium with or without 150 mM mannitol for further growth. 7 days later, the heights of the plants were observed. Since the overexpression plants grew at a rate different from the control plants in the medium without mannitol, the relative plant height (i.e. the plant height in the medium with mannitol is divided by the plant height in the normal medium) was used to assess the resistance to osmosis stress of the plants. The relative height of the OsbZIP46CA1 overexpression plants was remarkably higher than that of the wild type and full length OsbZIP46 overexpression line (FIG. 6 and FIG. 7), showing the overexpression of OsbZIP46CA1 gene enhances the oasmosis stress resistance of the transgenic plants.

### Example 7

### The Expression of Downstream Genes in OsbZIP46CA1 Overexpression Plants Upon Induction

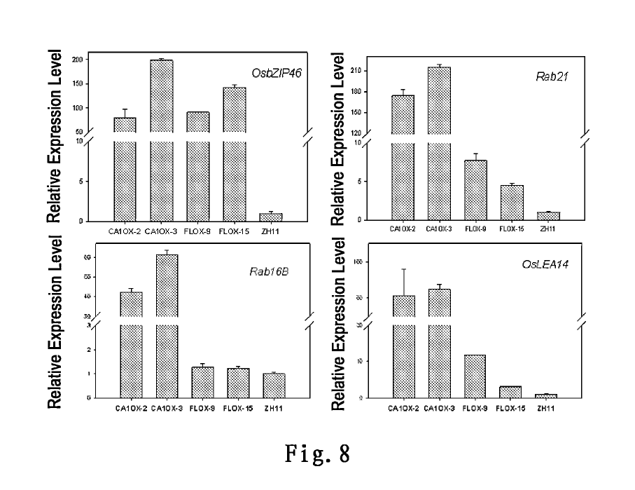

The expression amounts of three downstream genes and OsbZIP46 per se in OsbZIP46CA1 overexpression plants were measured by fluorescent real time quantitative PCR method (as described in Example 3). Two OsbZIP46CA1 overexpression lines (CA1OX-2, CA1OX-5) were measured with two full length OsbZIP46 overexpression lines (FLOX-9, FLOX-15) and wild type (ZH11) as controls. The results demonstrated that the expression of OsbZIP46CA1 or OsbZIP46 gene in the two OsbZIP46CA1 overexpression lines and the two full length OsbZIP46 overexpression lines was essentially at the same level, while the expression of downstream genes were strongly induced in OsbZIP46CA1 overexpression lines compared with that in wild type; but the expression of downstream genes were slightly induced or essentially not induced in the full length OsbZIP46 overexpression lines compared with that in wild type, wherein most of these downstream genes are involved in drought response. This shows that the overexpression of OsbZIP46CA1 can enhance the drought response of plants, instead of that of full length OsbZIP46 (FIG. 8).

1-19. (canceled)

20. A recombinant polynucleotide encoding a polypeptide exhibiting constitutive transcriptional activation activity, wherein said polypeptide is an OsbZIP46 protein lacking domain D thereof.

21. The recombinant polynucleotide of claim 20, wherein said polypeptide comprises the amino acid sequence as set forth in SEQ ID NO:2.

22. The recombinant polynucleotide of claim 21, wherein said polynucleotide comprises the nucleotide sequence as set forth in SEQ ID NO:1.

23. A DNA construct comprising a plant active promoter operably linked to the recombinant polynucleotide of claim 20.

24. The DNA construct of claim 23, wherein said plant active promoter is a drought inducible promoter.

25. A transgenic plant or plant part comprising the DNA construct of claim 23, wherein said plant is drought tolerant and said plant part comprises seed or cell.

26. The transgenic plant or plant part of claim 25, wherein said plant is selected from the group consisting of a corn, cotton, soybean, rice and wheat plant.

27. A method of improving drought resistance of a plant, comprising providing a transgenic plant comprising the DNA construct of claim 23, wherein expression of said polynucleotide is induced under drought condition thereby improving the drought resistance of the transgenic plant.

28. The method of claim 27, wherein said providing step comprises transforming a plant with said DNA construct thereby obtaining said transgenic plant.

29. The method of claim 27, wherein said plant is selected from the group consisting of a corn, cotton, soybean, rice and wheat plant.

30. A method of sowing, planting, or growing a plant with improved drought resistance, said method comprising the step of sowing, planting, or growing a transgenic plant comprising the DNA construct of claim 23.

31. A DNA construct comprising a heterologous promoter operably linked to the recombinant polynucleotide of claim 20, wherein said promoter is an ubiquitin promoter.

32. The DNA construct of claim 31, wherein said promoter is a corn ubiquitin promoter.

33. A transgenic plant or plant part comprising the DNA construct of claim 31, wherein said plant is drought tolerant and said plant part comprises seed or cell.

34. The transgenic plant or plant part of claim 33, wherein said plant is selected from the group consisting of a corn, cotton, soybean, rice and wheat plant.

35. A method of improving drought resistance of a plant, comprising providing a transgenic plant comprising the DNA construct of claim 31, wherein said polynucleotide is overexpressed compared to a wild type plant, said transgenic plant exhibiting improved drought resistance compared to the wild type plant.

36. The method of claim 35, wherein said providing step comprises transforming a plant with said DNA construct thereby obtaining said transgenic plant.

37. The method of claim 35, wherein said plant is selected from the group consisting of a corn, cotton, soybean, rice and wheat plant.

38. A method of sowing, planting, or growing a plant with improved drought resistance, said method comprising the step of sowing, planting, or growing a transgenic plant comprising the DNA construct of claim 31.


SEQUENCE LISTING (20 sequences)

SEQ ID NO: 1 - DNA, 678 bp, Artificial sequence
  CDS (1)..(678): DNA sequence encoding modified OsbZIP46 (i.e. OsbZIP46CA1)
atggagttgccggcggatgggagcgcgctggcgaggcaggggtcgatctactcgctgacgttcgacgagttccagagcgcgctgggaagcgccgagaaggatttcgggtcgatgaacatggatgagctgctgcgcaacatctggacggcggaggagtcgcaggccatagcgccggcggcggcggctgcttcggcggcggcggtggttggggacgcgcagcagcagcagcagccgatccagaggcaggggtcgctgacgctgccacgcacgctgagccagaagacggtggacgaggtgtggcgcgacatcatgggcttgggcggcagcgacgacgaagaccccgcggcggcggcggctgcgtcgccggtgccttacccattcgacaccgcgctcagggtgaggaagggccctaccgtcgagaaggtggtggagaggcggcagaggcggatgatcaagaacagggagtccgctgctaggtctcgcgcgcggaagcaggcttatataatggagttggaagctgaggtggcaaaactgaaggaacagaaggctgaattgcagaaaaagcaggtggaaatgatacagaagcaaaatgatgaggtcatggagagaatcactcagcaacttggaccaaaggcaaagagattttgcctccgacgaacactgactggtccatgctga

SEQ ID NO: 2 - protein, 225 aa, Artificial sequence
  amino acid sequence of modified OsbZIP46 (i.e. OsbZIP46CA1)
MELPADGSALARQGSIYSLTFDEFQSALGSAEKDFGSMNMDELLRNIWTAEESQAIAPAAAAASAAAVVGDAQQQQQPIQRQGSLTLPRTLSQKTVDEVWRDIMGLGGSDDEDPAAAAAASPVPYPFDTALRVRKGPTVEKVVERRQRRMIKNRESAARSRARKQAYIMELEAEVAKLKEQKAELQKKQVEMIQKQNDEVMERITQQLGPKAKRFCLRRTLTGPC

SEQ ID NO: 3 - DNA, 17 bp, Artificial Sequence
  universal PCR primer
atttaggtgacactata

SEQ ID NO: 4 - DNA, 20 bp, Artificial Sequence
  universal PCR primer T7
taatacgactcactataggg

SEQ ID NO: 5 - DNA, 45 bp, Artificial Sequence
  forward primer OsbZIP46p32F1-attB1
ggacaagtttgtacaaaaaagcaggcttggagttgccggcggatg

SEQ ID NO: 6 - DNA, 47 bp, Artificial Sequence
  reverse primer OsbZIP46p32R1-attB2
ggaccactttgtacaagaaagctgggttcagcatggaccagtcagtg

SEQ ID NO: 7 - DNA, 48 bp, Artificial Sequence
  reverse primer OsbZIP46p32R2-attB2
ggaccactttgtacaagaaagctgggttcaagtgattctctccatgac

SEQ ID NO: 8 - DNA, 49 bp, Artificial Sequence
  reverse primer OsbZIP46p32R4-attB2
ggaccactttgtacaagaaagctgggttcactcgacggtagggcccttc

SEQ ID NO: 9 - DNA, 49 bp, Artificial Sequence
  reverse primer OsbZIP46p32R5-attB2
ggaccactttgtacaagaaagctgggttcacagcatcccgttggcgagc

SEQ ID NO: 10 - DNA, 48 bp, Artificial Sequence
  reverse primer OsbZIP46CA0R-attB2
ggaccactttgtacaagaaagctgggttcacatgtcctcccgcacgac

SEQ ID NO: 11 - DNA, 54 bp, Artificial Sequence
  reverse primer OsbZIP46p32R6-attB2
ggaccactttgtacaagaaagctgggttcacgcagccgccgccgccgcggggtc

SEQ ID NO: 12 - DNA, 51 bp, Artificial Sequence
  reverse primer OsbZIP46p32R7-attB2
ggaccactttgtacaagaaagctgggttcacgccgccggcgctatggcctg

SEQ ID NO: 13 - DNA, 24 bp, Artificial Sequence
  reverse primer OsbZIP46CuActR1
cgcagccgccgccgccgcggggtc

SEQ ID NO: 14 - DNA, 42 bp, Artificial Sequence
  forward primer OsbZIP46CuActF1
gaccccgcggcggcggcggctgcgtcgccggtgccttaccca

SEQ ID NO: 15 - DNA, 28 bp, Artificial Sequence
  forward primer OsbZIP46-F-Kpn1
ataggtaccatggagttgccggcggatg

SEQ ID NO: 16 - DNA, 29 bp, Artificial Sequence
  reverse primer OsbZIP46-R-BamH1
ataggatcctcagcatggaccagtcagtg

SEQ ID NO: 17 - DNA, 19 bp, Artificial Sequence
  forward primer OsbZIP46rtNTterminalF
aagcgccgagaaggatttc

SEQ ID NO: 18 - DNA, 17 bp, Artificial Sequence
  reverse primer OsbZIP46rtNTterminalR
ccgccgtccagatgttg

SEQ ID NO: 19 - DNA, 21 bp, Artificial Sequence
  forward primer actin76F
tggcatctctcagcacattcc

SEQ ID NO: 20 - DNA, 20 bp, Artificial Sequence
  reverse primer actin76R
tgcacaatggatgggtcaga